(19) United States
(12) Patent Application Publication
(10) Pub. No.: US 2021/0145561 A1
(43) Pub. Date: May 20, 2021

(54) SYSTEM FOR CEREBRAL EMBOLIC PROTECTION DURING HEART VALVE PROCEDURE

(71) Applicant: Bedrosian Global, LLC, Malibu, CA (US)

(72) Inventors: David Rizik, Scottsdale, AZ (US); Bert Bedrosian, Malibu, CA (US)

(21) Appl. No.: 16/953,887

(22) Filed: Nov. 20, 2020

Related U.S. Application Data
(60) Provisional application No. 62/938,145, filed on Nov. 20, 2019.
(60) Provisional application No. 62/955,209, filed on Dec. 30, 2019.

(51) Int. Cl. A61F 2/01; A61B 17/12; A61F 2/24

(52) U.S. Cl. CPC A61F 2/013; A61B 2017/1205; A61F 2/2433; A61B 17/1204

(57) ABSTRACT

A catheter assembly that includes a catheter, a heart valve procedure device positioned at a distal end of the catheter, and an embolic protection device positioned on the catheter proximal of the heart valve procedure device. The embolic protection device is movable between a stowed position and a deployed position.

### CROSS REFERENCE TO RELATED APPLICATIONS

This application claims the benefit of U.S. Provisional Application No. 62/938,145, filed Nov. 20, 2019, and U.S. Provisional Application No. 62/955,209, filed Dec. 30, 2019 the entireties of which are incorporated herein by reference.

### FIELD OF THE INVENTION

The present invention relates generally to a device for cerebral embolic protection, and more particularly to a device for cerebral embolic protection during a heart valve replacement procedure.

### BACKGROUND OF THE INVENTION

Transcatheter aortic valve replacement (TAVR) has become increasingly popular for replacing native heart valves in recent years. However, their delivery and implantation carries with it the risk of embolic material being carried away by the bloodstream and to the brain, which can cause a stroke. As a result, cerebral embolic protection (CEP) devices are provided to reduce the risk of stroke. However, CEP devices are delivered to the site where they are deployed through a different entry pathway than the TAVR. This requires two separate devices, and increases the risk of complications.

Percutaneous balloon aortic valvuloplasty (PBAV) is another known cardiac procedure that carries the risk of calcium breaking off and embolizing (traveling) to the brain. In PBAV a catheter is used to deliver a balloon to the native valve site to dilate the stenosed aortic valve. PBAV can be performed as a standalone procedure or prior to TAVR.

The background description disclosed anywhere in this patent application includes information that may be useful in understanding the present invention. It is not an admission that any of the information provided herein is prior art or relevant to the presently claimed invention, or that any publication specifically or implicitly referenced is prior art.

### SUMMARY OF THE PREFERRED EMBODIMENTS

In accordance with a first aspect of the present invention there is provided a catheter assembly that includes a catheter, a heart valve procedure device positioned at a distal end of the catheter, and an embolic protection device positioned on the catheter proximal of the heart valve procedure device. The embolic protection device is movable between a stowed position and a deployed position. In a preferred embodiment, the catheter assembly includes a switch member that is configured to move the embolic capture device between the stowed position and the deployed position. Preferably, the switch member is movable between a first position and a second position, and movement of the switch from the first position to the second position moves the embolic capture device from the stowed position to the deployed position.

In a preferred embodiment, the embolic protection device is an embolic capture device that is positioned to span the ascending aorta when in the deployed position. The embolic capture device is configured to capture embolic material. Preferably, the embolic capture device includes a filter portion and a plurality of stretcher members. The stretcher members are movable from a collapsed position to an expanded position to move the filter portion from the stowed position to the deployed position. In a preferred embodiment, the stretcher members are movable within one or more slots defined a wall of the catheter. The One end of the slot represents or stops the stretcher member in the collapsed position, and the opposite end of the slot represents or stops the stretcher member in the expanded position.

In a preferred embodiment, the embolic protection device is an embolic deflection device that is positioned to occlude the aortic arch vessels when in the deployed position. The embolic deflection device is configured to deflect embolic material from the aortic arch vessels while allowing blood to perfuse therethrough (through the filter portion).

In a preferred embodiment, the catheter defines a first lumen and a second lumen therein. The first lumen is associated with the heart valve procedure device and the second lumen is associated with the embolic protection device. At least a portion of the components for deploying the heart valve procedure device extend through the first lumen and at least a portion of the components for deploying the embolic protection device extend through the second lumen. The heart valve procedure device can include a replacement heart valve and/or an expandable balloon, among other devices. The embolic protection device can be self-expanding.

In a preferred embodiment, the catheter assembly includes a switch member and at least a first runner member operatively disposed between the switch member and the stretcher members. Movement or actuation of the switch member from a first position to a second position (first and second position can apply to an electrical switch) moves the runner member within the catheter and moves the embolic capture device from the stowed position to the deployed position.

In a preferred embodiment, the embolic capture device includes a filter portion having a catheter opening defined therein. The catheter extends through the catheter opening. The filter portion is co-axial with the catheter.

In accordance with another aspect of the present invention there is provided a method performed in a heart that includes a heart valve that includes the steps of (a) obtaining a catheter assembly that includes a heart valve procedure device positioned at a distal end of a catheter, and an embolic protection device positioned on the catheter proximal of the heart valve procedure device, (b) maneuvering the catheter assembly to a position such that the heart valve procedure device is positioned adjacent the heart valve and the embolic protection device is positioned proximal the heart valve procedure device, (c) moving the embolic protection device from a stowed position to a deployed position, and (d) deploying the heart valve procedure device. In a preferred embodiment, the catheter defines a first lumen and a second lumen therein and the performance of step (d) is associated with the first lumen and the performance of step (c) is associated with the second lumen. It will be appreciated that in a TAVR procedure, the embolic protection device is positioned and configured to prevent embolic material from entering the aortic arch vessels.

The present invention is the combination of a cerebral embolic protection (CEP) device in combination with a heart valve procedure device (replacement heart valve, balloon, balloon expandable valve, self-expanding replacement heart valve) that is delivered via a catheter.

In a preferred embodiment, the present invention provides a CEP device together with a transcatheter aortic valve replacement valve (a TAVR) as a single unit. The CEP device can be a polyurethane 130 to 150u filter embedded in or “preloaded” on a TAVR device. In a preferred embodiment, the CEP/TAVR system comes as a single unit and the CEP can be deployed and retrieved during the TAVR procedure.

In another embodiment the heart valve procedure device is a percutaneous balloon aortic valvuloplasty (PBAV) balloon that is provided together with a CEP device or system. The embolization protection “filter” device can be attached to or part of the the balloon catheter used in PBAV and can be positioned several centimeters above the balloon in the ascending aorta. This allows for capturing of calcium and other debris which might come off the valve and prevent it from going to the brain. Accordingly, the filter or CEP device can be delivered as a unit with a balloon used for PBAV or a TAVR device.

CEP devices for use during the performance of cardiac procedures and delivered by catheter are known. For example, see US Publication No. 2001/0020160 and US Publication No. 2016/0151141, the entireties of which are incorporated by reference herein.

### BRIEF DESCRIPTION OF THE DRAWINGS

The invention may be more readily understood by referring to the accompanying drawings in which:

FIG. 1 is a perspective view of a catheter assembly positioned within the aorta and that includes a heart valve procedure device and an embolic capture device in accordance with a preferred embodiment of the present invention and showing the embolic capture device in the stowed position;

FIG. 2 is a perspective view of the catheter assembly showing the embolic capture device in the deployed position;

FIG. 3 is a cross-sectional view of a catheter with dual lumina;

FIG. 4A is a perspective view of a portion of the catheter assembly showing an embodiment of a switch member;

FIG. 4B is a perspective view of a portion of the catheter assembly showing an embodiment of a switch member;

FIG. 5 is a perspective view of the catheter assembly showing the embolic capture device in the deployed position and the heart valve procedure device as an expandable balloon;

FIG. 6 is a perspective view of a catheter assembly positioned within the aorta and that includes a heart valve procedure device and an embolic deflection device in accordance with a preferred embodiment of the present invention and showing the embolic deflection device in the deployed position; and

FIG. 7 is a perspective view of the catheter assembly of FIG. 6 where the embolic deflection device is self-expanding as it is released from a sheath.

Like numerals refer to like parts throughout the several views of the drawings.

### DETAILED DESCRIPTION OF THE PREFERRED EMBODIMENTS

The following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be, but not necessarily are references to the same embodiment; and, such references mean at least one of the embodiments. If a component is not shown in a drawing then this provides support for a negative limitation in the claims stating that that component is “not” present. However, the above statement is not limiting and in another embodiment, the missing component can be included in a claimed embodiment.

Reference in this specification to “one embodiment,” “an embodiment,” “a preferred embodiment” or any other phrase mentioning the word “embodiment” means that a particular feature, structure, or characteristic described in connection with the embodiment is included in at least one embodiment of the-disclosure and also means that any particular feature, structure, or characteristic described in connection with one embodiment can be included in any embodiment or can be omitted or excluded from any embodiment. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others and may be omitted from any embodiment. Furthermore, any particular feature, structure, or characteristic described herein may be optional. Similarly, various requirements are described which may be requirements for some embodiments but not other embodiments. Where appropriate any of the features discussed herein in relation to one aspect or embodiment of the invention may be applied to another aspect or embodiment of the invention. Similarly, where appropriate any of the features discussed herein in relation to one aspect or embodiment of the invention may be optional with respect to and/or omitted from that aspect or embodiment of the invention or any other aspect or embodiment of the invention discussed or disclosed herein.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted.

It will be appreciated that the same thing can be said in more than one way. Consequently, alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” “aft,” “forward,” “inboard,” “outboard” and “below” used herein are merely for ease of description and refer to the orientation of the components as shown in the figures. It should be understood that any orientation of the components described herein is within the scope of the present invention.

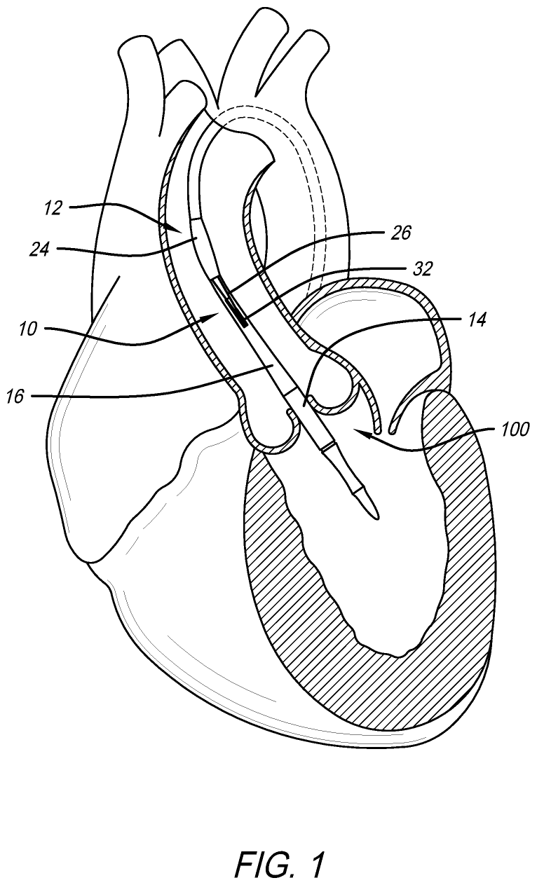

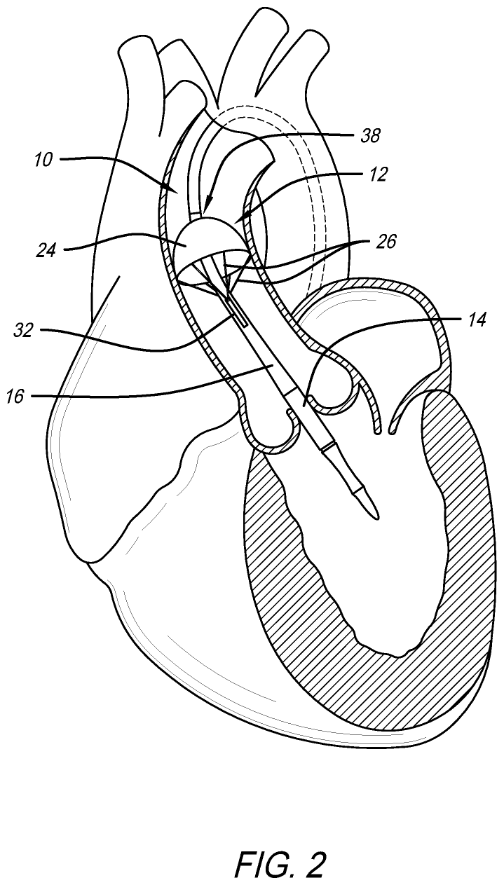

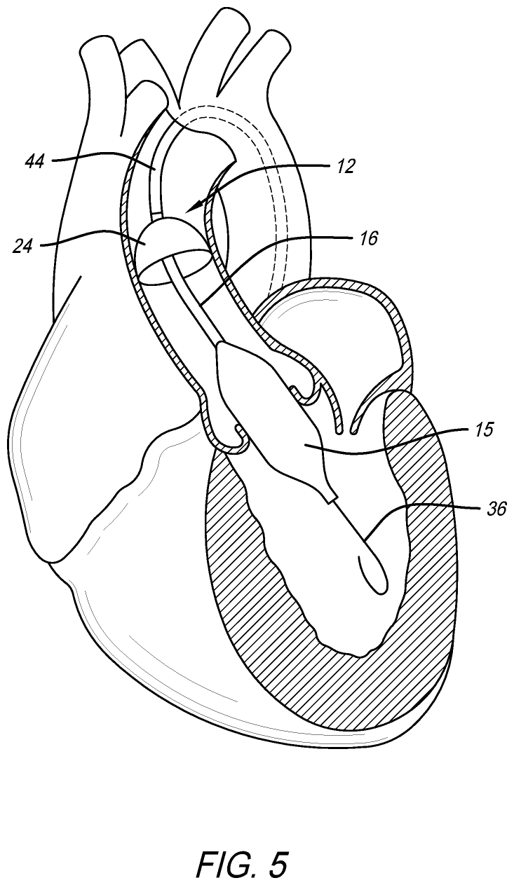

Generally, the present invention is the combination of a cerebral embolic protection device (CEP device or embolic protection device) in combination with another heart valve procedure device that are delivered together via a catheter. As shown in FIGS. 1-3, in a first preferred embodiment, the present invention is a catheter assembly 10 that includes an embolic capture device 12 (one type of embolic protection device) and a replacement heart valve 14 (see FIGS. 1 and 2), balloon 15 (see FIG. 5), balloon and replacement heart valve (for a balloon expandable valve) or other device that can be used or deployed with the heart. These devices are referred to together herein as a “heart valve procedure device” or a “cardiac-related device”. It will be appreciated that the embolic capture device 12 and replacement heart valve are both delivered by and may be deployed through or via a catheter 16 so that a transcatheter aortic valve replacement (TAVR) procedure can be performed. TAVR is used as example herein, but it should be understood that the present invention can be used for and in other procedures that include a heart valve procedure device or cardiac-related device and where protection from embolic material reaching the brain is desired.

FIG. 1 shows the replacement heart valve 14, positioned at the native heart valve 100, but prior to being deployed, together with the embolic capture device 12 prior to deployment. FIG. 1 also shows the deployment and retrieval profile of the embolic capture device 12 and the replacement heart valve 14. The catheter assembly 10 also includes a delivery member 18 on which the replacement heart valve 14 is positioned. As will be appreciated by those of ordinary skill in the art, the replacement heart valve 14 is positioned at or near the distal end of the delivery member 18 and the delivery member 18 is moved out of the catheter 16 once it is properly positioned within the aorta (or other location in the heart for other procedures).

As shown in FIG. 1, the present invention provides the embolic capture device 12 together with the replacement heart valve 14 as a single unit. The embolic capture device 12 can be a polyurethane 130 to 150u filter or the like that spans the ascending aorta to prevent embolic material from reaching the aortic arch vessels, and ultimately, the brain. The embolic capture device 12 is movable between a stowed position (see FIG. 1) and a deployed position (see FIG. 2). In the stowed position, the embolic capture device 12 is movable through the lumen of the catheter 16. In the deployed position, the embolic capture device 12 is positioned to capture embolic material caused by a heart valve procedure using a heart valve procedure device (in FIGS. 1-2, the replacement heart valve 14). Any mechanism or method for moving the embolic capture device 12 between the stowed and deployed positions is within the scope of the present invention.

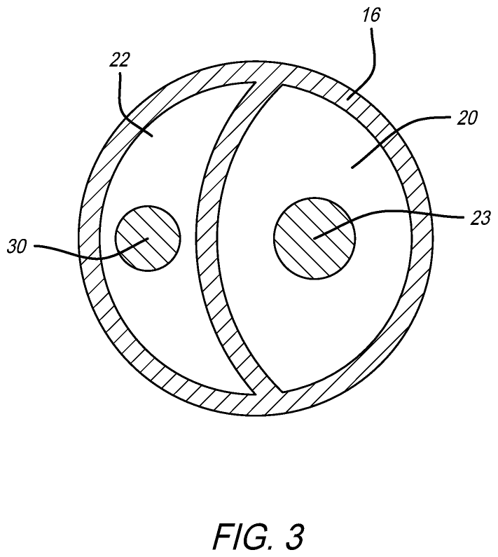

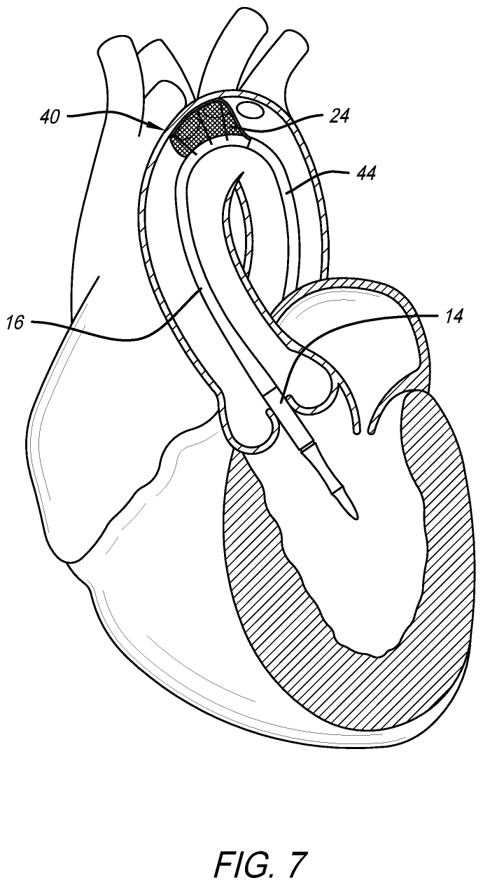

As shown in FIG. 3, the replacement heart valve 14 (or other cardiac-related device) can extend through or be delivered through a first lumen 20 defined within the catheter 16 and the embolic capture device 12 (or other embolic protection device) can be delivered through or operated within a second lumen 22 defined within the catheter 16. It will be appreciated by those of ordinary skill in the art that, based on the heart valve procedure device being used, the components within the catheter lumen will be different. Therefore, for simplicities sake, component 23 is intended to represent any of the different components for delivering, operating, filling, manipulating, controlling or the like the heart valve procedure device. Component 23 is referred to herein as a heart valve procedure device deployment member 23. For example, in a preferred embodiment, the delivery member may be a tubular member on which the replacement heart valve is crimped and through which a guide wire extends. FIG. 3 shows the first lumen 20 and the second lumen 22 side by side. However, this is not a limitation on the present invention. In another embodiment, the first and second lumina can be co-axial. In another embodiment, both the heart valve procedure device and the embolic protection device can be delivered through or operated within a common lumen. In another embodiment, only the heart valve procedure device is operated or deployed through a lumen in the catheter and the embolic protection device is deployed by unsheathing (e.g., pulling a sheath proximally to allow the embolic protection device to deploy or pushing the catheter out of the sheath to allow the embolic protection device to deploy (see. FIG. 7).

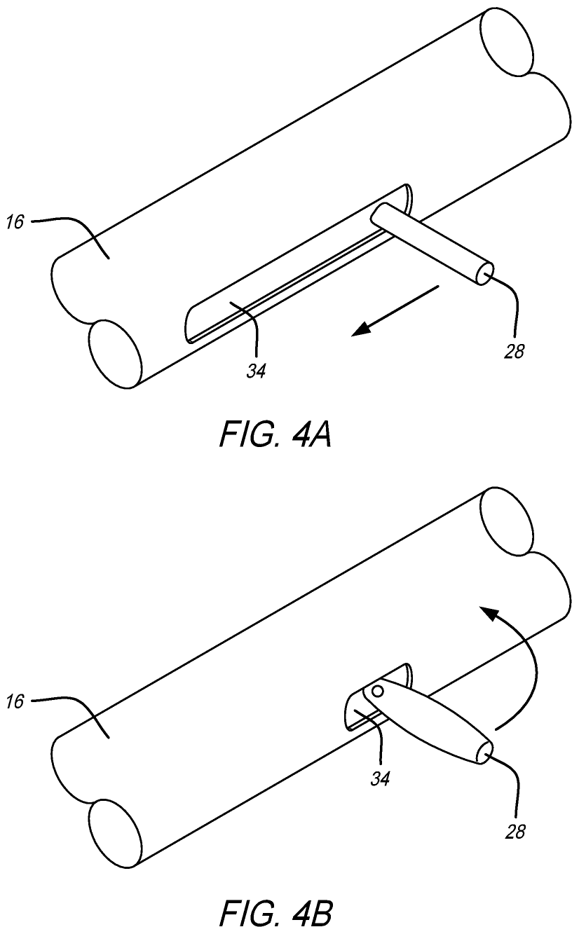

In a preferred embodiment, as shown in FIG. 2, the embolic capture device 12 includes a filter portion 24 and a plurality of stretcher members 26. The filter portion 24 The stretcher members 26 stretch or move the filter portion between the stowed and deployed positions (similar to an umbrella). The stretcher members 26 are operatively connected to a switch member 28 (see FIGS. 4A and 4B) that can be operated by a user at or near the proximal end of the catheter assembly 10 or the catheter 16. The switch member 28 can be a rod, a lever, a switch, a button or the like. One or more embolic protection device deployment members or runner members 30 are operatively disposed between the switch member 28 and the stretcher members 26. Movement of the switch member 28 from a first position to a second position moves the runner member 30 within the catheter 16, which causes the stretcher members 26 to move the filter portion 24 from the stowed position to the deployed position. The stretcher members 26 may extend through one or more slots 32 defined in the wall of the catheter 16 so that they can operatively connect to the runner member(s) 30. FIGS. 1 and 2 show a slot 32 that receives the ends of two of the stretcher members 26. The stretcher members 26 move along the slot 32 between a first end and a second end. The stretcher members 26 may all be attached to a single runner member 30 (which may be a cord, cable or the like) or each stretcher member 26 may be connected to a single runner member 30.

As shown in FIG. 2, in a preferred embodiment, the catheter 16 extends through the embolic capture device 12 and, more specifically, the filter portion 24. The filter portion 24 may have a catheter opening 38 defined therein through which the catheter 16 extends.

In the embodiment shown in FIG. 4A, the switch member 28 is a rod that is moved within a slot 34 or opening in the catheter wall between the first position (shown in FIG. 4A) and the second position, such that the filter portion 24 is moved from the stowed position to the deployed position. FIG. 4A shows that the operator would pull the switch member 28 toward the proximal end of the catheter assembly 10 to deploy the filter portion. In another embodiment, the switch member 28 can be pushed. In the embodiment shown in FIG. 4B, the switch member 28 is a lever that is pivoted between the first position (shown in FIG. 4A) and the second position (see the curved arrow). These are examples of how the embolic capture device can be deployed. The switch member can also be located separate from the catheter. For example, the switch member can include a wire, tube or the like that runs to the catheter. In another embodiment, the embolic capture device 12 can be self-expanding.

FIG. 5 shows another preferred embodiment of the present invention, a balloon 15 (e.g., a PBAV balloon) in combination with the embolic capture device 12. In this embodiment, the tubular member on which the balloon 15 is disposed moves over a guidewire 36, as is known in the art. The embolic capture device 12 in this embodiment is self-expanding. In other words, the embolic capture device 12 is made of a material, such as nitinol that causes the embolic capture device 12 to expand to the deployed position once is comes out of the catheter 16, sheath or other tubular member. When the embolic capture device 12 is pulled back into the catheter or sheath, the embolic capture device 12 is collapsed back to the stowed position.

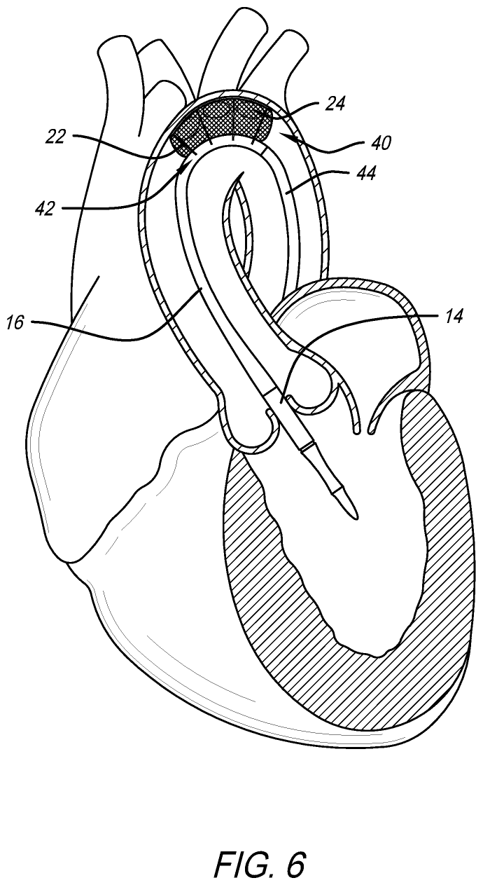

With reference to FIGS. 6 and 7, in another embodiment, the embolic protection device is an embolic deflection device 40 that is positioned to occlude or block the aortic arch vessels. The embolic deflection device 40 can be controlled or deployed similar to the embolic capture device 12 discussed herein. Therefore, all disclosure related to the embolic capture device 12 applies to the embolic deflection device 40. FIGS. 6 and 7 show the embolic deflection device 40 as a shield that has a generally U-shaped cross-section. In another embodiment, the embolic deflection device 40 can be a full tube with a circular or fully enclosed cross-section. The embolic deflection device 40 is movable between a stowed position where is at least partially wraps around or surrounds the catheter 16 and a deployed position, as shown in FIG. 6. In a preferred embodiment, the embolic deflection device 40 includes stretcher members 26 and a filter portion 24. The stretcher members 26 extend through openings 42 defined in the catheter wall that are operatively connected to a deployment member at the proximal end of the catheter assembly. One or more runner members extend through the lumen of the catheter. Movement of the deployment member from the first position to the second position causes the embolic deflection device 40 to move from the stowed position to the deployed position.

FIG. 7 shows a self-expanding embolic deflection device 40. In this embodiment, when the catheter 16 and embolic deflection device 40 move out of the sheath 44 or other sleeve or tubular member, the embolic deflection device 40 self-expands from the stowed position to the deployed position.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. Further, any specific numbers noted herein are only examples: alternative implementations may employ differing values, measurements or ranges.

Although the operations of any method(s) disclosed or described herein either explicitly or implicitly are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

The teachings of the disclosure provided herein can be applied to other systems, not necessarily the system described above. The elements and acts of the various embodiments described above can be combined to provide further embodiments. Any measurements or dimensions described or used herein are merely exemplary and not a limitation on the present invention. Other measurements or dimensions are within the scope of the invention.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

While certain aspects of the disclosure are presented below in certain claim forms, the inventors contemplate the various aspects of the disclosure in any number of claim forms. For example, while only one aspect of the disclosure is recited as a means-plus-function claim under 35 U.S.C. § 112, ¶6, other aspects may likewise be embodied as a means-plus-function claim, or in other forms, such as being embodied in a computer-readable medium. (Any claims intended to be treated under 35 U.S.C. § 112, ¶6 will include the words “means for”). Accordingly, the applicant reserves the right to add additional claims after filing the application to pursue such additional claim forms for other aspects of the disclosure.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A catheter assembly comprising:
a catheter,
a heart valve procedure device positioned at a distal end of the catheter, and
an embolic protection device positioned on the catheter proximal of the heart valve procedure device, wherein the embolic protection device is movable between a stowed position and a deployed position.

2. The catheter assembly of claim 1 further comprising a switch member configured to move the embolic capture device between the stowed position and the deployed position.

3. The catheter assembly of claim 2 wherein the switch member is movable between a first position and a second position, and wherein movement of the switch from the first position to the second position moves the embolic capture device from the stowed position to the deployed position.

4. The catheter assembly of claim 1 wherein the embolic protection device is an embolic capture device that is positioned to span the ascending aorta when in the deployed position, wherein the embolic capture device is configured to capture embolic material.

5. The catheter assembly of claim 4 wherein the embolic capture device includes a filter portion and a plurality of stretcher members, wherein the stretcher members are movable from a collapsed position to an expanded position to move the filter portion from the stowed position to the deployed position.

6. The catheter assembly of claim 4 wherein the embolic protection device is an embolic deflection device that is positioned to occlude the aortic arch vessels when in the deployed position, wherein the embolic deflection device is configured to deflect embolic material.

7. The catheter assembly of claim 1 wherein the catheter defines a first lumen and a second lumen therein, wherein the first lumen is associated with the heart valve procedure device and the second lumen is associated with the embolic protection device.

8. The catheter assembly of claim 1 wherein the heart valve procedure device includes a replacement heart valve.

9. The catheter assembly of claim 1 wherein the heart valve procedure device includes an expandable balloon.

10. The catheter assembly of claim 1 wherein the embolic protection device is self-expanding.

11. The catheter assembly of claim 5 further comprising a switch member and at least a first runner member operatively disposed between the switch member and the stretcher members, wherein movement of the switch member from a first position to a second position moves the runner member within the catheter and moves the embolic capture device from the stowed position to the deployed position.

12. The catheter assembly of claim 5 wherein the stretcher members are movable within one or more slots defined a wall of the catheter.

13. The catheter assembly of claim 4 wherein the embolic capture device includes a filter portion, wherein a catheter opening is defined in the filter portion, and wherein the catheter extends through the catheter opening.

14. A catheter assembly comprising:
a catheter,
a heart valve procedure device positioned at a distal end of the catheter,
an embolic capture device positioned on the catheter proximal of the heart valve procedure device, wherein the embolic capture device is movable between a stowed position and a deployed position, wherein the embolic capture device includes a filter portion and a plurality of stretcher members, wherein the stretcher members are movable from a collapsed position to an expanded position to move the filter portion from the stowed position to the deployed position, wherein a catheter opening is defined in the filter portion, wherein the catheter extends through the filter portion, wherein the catheter defines a first lumen and a second lumen therein, wherein the first lumen is associated with the heart valve procedure device and the second lumen is associated with the embolic capture device.

15. A method performed in a heart that includes a heart valve, the method comprising the steps of:
(a) obtaining a catheter assembly that includes a heart valve procedure device positioned at a distal end of a catheter, and an embolic protection device positioned on the catheter proximal of the heart valve procedure device,
(b) maneuvering the catheter assembly to a position such that the heart valve procedure device is positioned adjacent the heart valve and the embolic protection device is positioned proximal the heart valve procedure device,
(c) moving the embolic protection device from a stowed position to a deployed position, and
(d) deploying the heart valve procedure device.

16. The method of claim 15 wherein the catheter defines a first lumen and a second lumen therein, wherein the performance of step (d) is associated with the first lumen and the performance of step (c) is associated with the second lumen.
